(19) United States
(12) Patent Application Publication
(10) Pub. No.: US 2026/0355204 A1
(43) Pub. Date: Nov. 12, 2026

(54) TOPICAL BRAF INHIBITOR COMPOSITIONS FOR TREATMENT OF ADVERSE CUTANEOUS REACTIONS INDUCED BY INHIBITOR AGENTS ACTING ON THE MITOGEN-ACTIVATED PROTEIN KINASE PATHWAY

(71) Applicant: Lutris Pharma Ltd., Tel Aviv (IL)

(72) Inventor: Noa Shelach, Tel Aviv (IL)

(21) Appl. No.: 19/658,965

(22) Filed: Apr. 27, 2026

Related U.S. Application Data
(60) Provisional application No. 63/795,034, filed on Apr. 25, 2025.

(51) Int. Cl. A61K 31/52; A61K 9/00; A61K 31/506; A61K 31/517; A61P 17/10

(52) U.S. Cl. CPC A61K 31/52; A61K 9/0014; A61K 31/506; A61K 31/517; A61P 17/10

(57) ABSTRACT

Pharmaceutical topical compositions comprising at least one compound of Formula I-III and method of using I-III same compositions for the treatment, amelioration or prevention of an EGFR, RAS, RAF, ERK, PI3K, MEK, SHP or HER inhibitor (each alone or in combination with another inhibitor), induced cutaneous adverse effects like acneiform rash in a subject in need thereof are provided. Said treatment has the effect of improving the adherence of said subject to the treatment, while preventing delays, discontinuation or interruption of the treatment or undesirable reduction of the label recommended inhibitor dosage.

The compounds of Formula I-III are effective for prevention, amelioration or treatment of cutaneous adverse effects at all MAPK pathway levels. The treatment is successful in ameliorating the severity of the acneiform rash by at least by one unit as well as preventing escalation of the cutaneous adverse effects.

### CROSS-REFERENCE TO RELATED APPLICATIONS

This application claims the benefit of U.S. Provisional Application 63/795,034, filed Apr. 25, 2025, the entire contents of which are incorporated herein by reference.

### FIELD OF THE INVENTION

The present disclosure relates to topical compositions comprising a compound of Formula I-IV and methods of treating cutaneous adverse reactions, in particular cutaneous reactions like acneiform rash, induced by an EGFR, RAS, ERK, PI3K, MEK, SHP or HER inhibitor (each alone or in combination with another inhibitor), by topical treatment of a subject in need thereof with said topical compositions.

### BACKGROUND

MAPK components inhibitors (in particular EGFR and K-RAS inhibitors) are increasingly used in the treatment of various diseases, in particular, in several types of cancers such as solid tumors with various mutations in the components of this pathway (Pipeline, Verastem, 2024).

### SUMMARY OF THE INVENTION

Topical compositions comprising compound of Formula I-IV and method of using composition comprising compound of Formula I-IV for the treatment, amelioration or prevention of cutaneous adverse effects induced by an EGFR, RAS, ERK, PI3K, MEK, SHP or HER inhibitor (each alone or in combination with another inhibitor) in a subject in need thereof are provided.

In one embodiment, a method of treating and/or preventing an EGFR, RAS, ERK, PI3K, MEK, SHP or HER inhibitor (each alone or in combination with another inhibitor)-induced acneiform lesions in a subject in need thereof is provided, the method comprising administering once a day for 4 to 12 weeks to the skin of the subject in need thereof a topical pharmaceutical composition comprising from about 0.01% weight/weight to about 0.3% weight/weight of a compound of Formula I-IV and a pharmaceutically acceptable carrier or excipient to a subject in need therefore for treating an EGFR, RAS, ERK, PI3K, MEK, SHP or HER inhibitor (each alone or in combination with another inhibitor)-induced acneiform lesions. In one embodiment, the topical pharmaceutical composition is applied for four to twelve weeks to the skin of the subject in need thereof.

In one embodiment, the topical composition comprises from about 0.01% to about 0.25% weight/weight of the compound of Formula I-IV. In one embodiment, the topical composition comprises about 0.01% weight/weight of the compound of Formula I-IV. In one embodiment, the topical composition comprises about 0.03% weight/weight of the compound of Formula I-IV. In one embodiment, the topical composition comprises about 0.1% weight/weight of the compound of Formula I-IV. In one embodiment, the topical composition comprises about 0.25% weight/weight of the compound of Formula I-IV. Each embodiment is a separate invention.

In one embodiment, the topical pharmaceutical composition is in the form of a gel, a hydrogel, an ointment, a cream, a spray, a dermal patch, a foam, a lotion or a liquid. In one embodiment, the topical pharmaceutical composition is in the form of a gel. In one embodiment, the topical pharmaceutical composition is in the form of a spray.

In one embodiment, the adverse cutaneous reactions are induced by inhibitor agents inhibiting the MAPK pathway downstream to EGFR. In one embodiment, the subject in need thereof has a cancer treated with an inhibitor agent, which inhibits the MAPK pathway downstream to EGFR.

In one embodiment, the inhibitor agents inhibiting the MAPK pathway downstream to EGFR are selected from an EGFR, RAS, ERK, PI3K, MEK, SHP or HER inhibitor (each alone or in combination with another inhibitor)

In one embodiment, the topical treatment of a subject in need thereof with a composition comprising at least one of the compounds of Formula I-IV in a therapeutically effective amount will bring about the reactivation of the MAPK pathway, with the net result of treatment, amelioration, reduction of the cutaneous adverse reaction grade with at least one grade and prevention of the escalation (or worsening) of the cutaneous adverse effects.

In one embodiment, the adverse cutaneous reactions are selected from acneiform rash, paronychia, xerosis, abnormal scalp, facial hair and/or eyelash growth, maculopapular rash, mucositis, post inflammatory hyperpigmentation and combinations thereof,

Acneiform rash is also known as acneiform eruptions, acneiform lesions or acne-like dermatoses,

In one embodiment the adverse cutaneous reactions are acneiform lesions. In one embodiment, the subject in need thereof has a cancer treated with an EGFR inhibitor. In one embodiment, the acneiform lesions are EGFR inhibitor-induced acneiform lesions. In one embodiment, the subject in need thereof has a cancer treated with a RAS inhibitor.

In one embodiment, the acneiform lesions are RAS inhibitor-induced acneiform lesions.

In one embodiment, the acneiform lesions are ERK inhibitor induced acneiform lesions. In one embodiment, the subject in need thereof has a cancer treated with an ERK inhibitor. Each embodiment is a separate invention.

In one embodiment, the acneiform lesions are SHP inhibitor induced acneiform lesions. In one embodiment, the subject in need thereof has a cancer treated with an SHP inhibitor. Each embodiment is a separate invention.

In one embodiment, the acneiform lesions are MEK inhibitor induced acneiform lesions. In one embodiment, the subject in need thereof has a cancer treated with an MEK inhibitor. Each embodiment is a separate invention.

In one embodiment, the subject in need thereof has grade 1 acneiform lesions. In one embodiment, the subject in need thereof has grade 2 acneiform lesions. In one embodiment, the subject in need thereof has grade 3 acneiform lesions. In one embodiment, the subject in need thereof has grade 4 acneiform lesions.

In one embodiment, the method reduces the severity or prevents escalation of the acneiform lesions. In one embodiment, the method reduces the severity of the acneiform lesions from grade 4 to grade 3, 2, 1, or 0. In one embodiment, the method reduces the severity of the acneiform lesions from grade 3 to grade 2, 1, or 0. In one embodiment, the method reduces the severity of the acneiform lesions from grade 2 to grade 1, or 0. In one embodiment, the method reduces the severity of the acneiform lesions from grade 1 to grade 0.

In one embodiment, the acneiform lesions are induced by treatment with RAS inhibitors as a treatment side-effect. The net result of the acneiform rash is compromised patient compliance (adherence to treatment) at the label recommended RAS inhibitor dose or worse, treatment discontinuation or delays due to the need for an undesirable RAS inhibitor dose reduction. In one embodiment, administration of a therapeutically effect amount of a compound of Formula I-IV prevents delay, discontinuation, or interruption of the treatment with RAS inhibitors.

In one embodiment, the acneiform lesions are induced by treatment with EGFR inhibitors as a treatment side-effect. The net result of the acneiform rash is compromised patient compliance (adherence to treatment) at the label recommended EGFR inhibitor dose or worse, treatment discontinuation or delays due to the need for an undesirable EGFR inhibitor dose reduction. In one embodiment, administration of a therapeutically effect amount of a compound of Formula I-IV prevents delay, discontinuation, or interruption of the treatment with EGFR inhibitors.

In one embodiment, the acneiform lesions are induced by treatment with ERK inhibitors as a treatment side-effect. The net result of the acneiform rash is compromised patient compliance (adherence to treatment) at the label recommended ERK inhibitor dose or worse, treatment discontinuation or delays due to the need for an undesirable ERK inhibitor dose reduction. In one embodiment, administration of a therapeutically effect amount of a compound of Formula I-IV prevents delay, discontinuation, or interruption of the treatment with ERK inhibitors.

In one embodiment, the acneiform lesions are induced by treatment with SHP inhibitors as a treatment side-effect. The net result of the acneiform rash is compromised patient compliance (adherence to treatment) at the label recommended SHP inhibitor dose or worse, treatment discontinuation or delays due to the need for an undesirable SHP inhibitor dose reduction. In one embodiment, administration of a therapeutically effect amount of a compound of Formula I-IV prevents delay, discontinuation, or interruption of the treatment with SHP inhibitors.

In one embodiment, the topical composition comprises about 0.01% w/w of the compound of Formula I-IV.

In one embodiment, the topical composition comprises about 0.25% w/w of the compound of Formula I-IV.

In one embodiment, the composition is in the form of a gel, a hydrogel, an ointment, a cream, a foam, a spray, a lotion, a liquid or a dermal patch.

In one embodiment, the topical composition comprises from about 0.01% to about 0.2% weight/weight of the compound of Formula I-IV.

Aspects of the disclosure relate to a topical pharmaceutical composition comprising from about 0.03% weight/weight to about 0.1% weight/weight of a compound of Formula I, when
(R=3-(Trifluoromethoxy)phenyl)
and a pharmaceutically acceptable carrier or excipient for use in a method of treating and/or preventing an EGFR, RAS, ERK, PI3K, MEK, SHP or HER inhibitor (each alone or in combination with another inhibitor)-induced acneiform lesions in a subject in need thereof.

Aspects of the disclosure relate to a topical pharmaceutical composition comprising from about 0.01% weight/weight to about 0.3% weight/weight of a compound of Formula I, preferably from about 0.03% w/w to about 0.1% w/w and a pharmaceutically acceptable carrier or excipient for use in a method of treating and/or preventing EGFR, RAS, ERK, PI3K, MEK, SHP or HER inhibitor (each alone or in combination with another inhibitor), -induced acneiform lesions in a subject in need thereof, wherein the topical pharmaceutical composition is administered topically for four to twelve weeks to the skin of the subject in need thereof.

### BRIEF DESCRIPTION OF THE DRAWINGS

FIG. 1 depicts the members of the MAPK pathway downstream to EGFR.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a,” “an,” and “the” include plural references unless the context clearly dictates otherwise.

The term “about” when used before a numerical designation, e.g., temperature, time, amount, and concentration, including range, indicates approximations which may vary by (+) or (−) 10%, 5% or 1%.

The terms “pharmaceutically acceptable salts” or “salts thereof” mean salts which are pharmaceutically acceptable, and which possess the desired pharmacological activity.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The term “carrier” as used herein, refers to nontoxic chemical compounds or agents that facilitate the incorporation of a compound into cells, e.g., dermal cells, or tissues. Carriers useful herein include any such materials known in the art, which are nontoxic and do not interact with other components of the formulation in which it is contained in a deleterious manner. As used herein, “pharmaceutically acceptable carrier” includes any and all solvents, dispersion media, and the like, which are pharmaceutically acceptable.

The term “therapeutically effective amount” of a compound or a composition refers to that amount of the compound or the composition that results in treatment, including reduction or inhibition of symptoms in a patient.

The term “subject” as used herein refers to organisms to be treated by the compounds or compositions of the present disclosure. Such organisms include a mammal, including a human or non-human mammal. The terms “patient”, “individual” and “subject” may be used interchangeably.

Provided herein are methods of treating RAS inhibitor, ERK inhibitor-induced cutaneous reactions using the compositions of the present disclosure.

The adverse effects of cutaneous adverse reactions to RAS or ERK- or other MAPK pathway inhibition include acneiform (papulopustular) rash, abnormal scalp, facial hair and/or eyelash growth, paronychia with or without pyogenic granulomas and telangiectasia.

Carnahan J. et al. (Mol Cancer Ther; 9(8) August 2010 p. 2399-2408, “Selective and Potent Raf Inhibitors Paradoxically Stimulate Normal Cell Proliferation and Tumor Growth”). found that in cells with wild-type B-Raf, exposure to Raf inhibitors resulted in a dose-dependent and sustained paradoxical activation of mitogen-activated protein kinase (MAPK) signaling. This paradoxical activation of MAPK pathway can be used for treating cutaneous adverse reactions induced by treatment with EGFR or PI3K inhibitors (see PCT/IL2017/050301 and PCT/IL2018/050836), and HER dimerization inhibitors (see Nami B et al. “The Effects of Pertuzumab and Its Combination with Trastuzumab on HER2 Homodimerization and Phosphorylation”. Cancers (Basel). 2019; 11(3):37; Nami B et al. “The Effects of Pertuzumab and Its Combination with Trastuzumab on HER2 Homodimerization and Phosphorylation”. Cancers, 77(3), 375, 2019, which are incorporated herein by reference in their entireties).

There is still a need in the art for the development of novel therapeutic compounds, compositions, and methods of treatment, to help alleviate the aforementioned cutaneous adverse reactions associated with administration of an EGFR, RAS, ERK, PI3K, MEK, SHP or HER inhibitors (each alone or in combination with another inhibitor), Provided herein are the compounds of Formula I-IV and compositions comprising them. Also provided are methods of treating cutaneous adverse reactions using the compounds and compositions comprising at least one of the compounds of Formula I-IV of the present disclosure.

The cutaneous adverse reactions (or lesions) are selected from acneiform rash, paronychia, xerosis, abnormal scalp, facial hair and/or eyelash growth, maculopapular rash, mucositis, post-inflammatory hyperpigmentation and combinations thereof.

Acneiform rash is also known as acneiform eruptions, acneiform lesions or acne-like dermatoses.

These adverse events can impair the patient's quality of life and adherence to treatment and in severe cases may require dose reduction or even temporary/permanent interruption of the anti-cancer therapy (Boone S L, Rademaker A, Liu D, et al. Impact and management of skin toxicity associated with anti-epidermal growth factor receptor therapy: survey results. Oncology. 2007; 72:152).

The adverse events caused by the treatment of a cancer subject in need thereof with EGFR inhibitor, RAS inhibitors, ERK inhibitors, PI3K inhibitors, MEK inhibitors, and/or HER inhibitors, alone or possibly in combination with an EGFR inhibitor can impair the patient's quality of life and adherence to treatment and in severe cases may require dose reduction or even temporary/permanent interruption of therapy (Boone S L, Rademaker A, Liu D, et al. Impact and management of skin toxicity associated with anti-epidermal growth factor receptor therapy: survey results. Oncology. 2007; 72:152.)

It occurred to the inventor of the instant disclosure, that the compounds of Formula I-IV may be effective for prevention, amelioration or treatment of cutaneous adverse effects not only at the level of EGFR, but also at the level of RAS and/or ERK, and/or SHP.

In one embodiment, provided herein is a method for treating, ameliorating and/or preventing an EGFR, RAS, ERK, PI3K, MEK, SHP or HER inhibitor (each alone or in combination with another inhibitor), induced acneiform lesions in a subject in need thereof, comprising administering a therapeutically effective amount of a composition comprising a compound of Formula I, Formula II, Formula III, Formula IV or a combination thereof and a pharmaceutically acceptable carrier or excipient.

In one embodiment, provided herein is a method for treating, ameliorating, and/or preventing a cutaneous adverse reaction of RAS inhibitors, EGFR inhibitors, ERK inhibitors, PI3K inhibitors, MEK inhibitors, SHP inhibitors, HER inhibitors or combinations thereof in a subject in need thereof, comprising administering a therapeutically effective amount of a composition comprising a compound of Formula I,

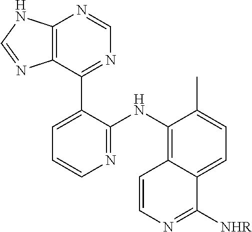

when
(R=3-(Trifluoromethoxy)phenyl)
and a pharmaceutically acceptable carrier or excipient.

In one embodiment, provided herein is a method for treating, ameliorating and/or preventing an EGFR, RAS, ERK, PI3K, MEK, SHP or HER inhibitor (each alone or in combination with another inhibitor), -induced acneiform lesions in a subject in need thereof, comprising administering a therapeutically effective amount of a composition comprising a compound of Formula I, when
(R=3-(Trifluoromethoxy)phenyl)
and a pharmaceutically acceptable carrier or excipient.

In one embodiment, methods for treating, ameliorating, and/or preventing a cutaneous adverse reaction of EGFR inhibitors, PI3K inhibitors, MEK inhibitors, HER dimerization inhibitors or combinations thereof in a subject in need thereof comprise administering topically a therapeutically effective amount of a topical pharmaceutical composition comprising a compound of Formula I, Formula II, Formula III, Formula IV or a combination thereof, or a pharmaceutically acceptable salt or a solvate thereof, or a combination thereof; and a pharmaceutically acceptable carrier or excipient.

It also occurred to the inventor of the present disclosure that besides the prevention, amelioration and treatment of the cutaneous lesions, the administration of the compositions comprising compounds of Formula I-IV has a no less important effect on the subject in need thereof.

As the cutaneous lesions become less of a bother, the adherence (patient compliance) of the subjects to the anti-cancer treatment is greatly improved.

Dermatological or cutaneous adverse reactions induced by anti-cancer treatment agents such as EGFR inhibitors, RAS inhibitors, ERK inhibitors, MEK inhibitors, SHP inhibitors, HER inhibitors or combinations thereof include acneiform rash, acneiform lesions, papulopustular rash, abnormal scalp hair growth, abnormal facial hair growth, abnormal hair growth, abnormal eyelash growth, xerosis, pruritus, paronychia with or without pyogenic granulomas and telangiectasia. The methods described herein treat, ameliorate, and/or prevent one or more of these adverse reactions.

In some embodiments, a cutaneous adverse reaction of RAS inhibitors, ERK inhibitors, MEK inhibitors, HER inhibitors or combinations thereof that is treated, ameliorated, and/or prevented by the compounds/compositions of the present disclosure is an acneiform rash (also referred herein as acneiform lesions).

In one embodiment, the subject is a mammal, human or non-human. In one embodiment, the subject is a human.

In one embodiment, the subject is receiving an EGFR, RAS, ERK, PI3K, MEK, SHP or HER inhibitor (each alone or in combination with another inhibitor), or a combination thereof at the time of the administration of the compounds/compositions of the present disclosure.

In one embodiment, the subject is receiving an EGFR inhibitor, RAS inhibitor, ERK inhibitor, MEK inhibitor, SHP inhibitor, HER inhibitor or a combination thereof preventively, before the administration of the compounds/compositions of the present disclosure.

In one embodiment, the subject is receiving an EGFR inhibitor, RAS inhibitor, ERK inhibitor, MEK inhibitor, SHP inhibitor, HER inhibitor or a combination thereof after the time of the administration of the compounds/compositions of the present disclosure and only if and when the cutaneous adverse effects of specifically acneiform lesions do appear.

In one embodiment, the subject has solid tumor, melanoma, lung cancer, head & neck cancer, colorectal cancer, brain cancer, multiple myeloma, breast cancer, pancreatic cancer, liver cancer or neurofibromatosis.

### Compounds

In one embodiment, provided herein is a compound of Formula I:

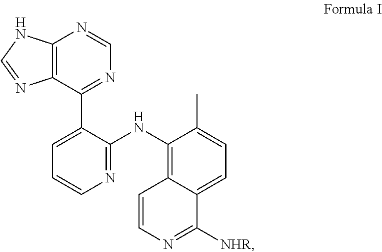

wherein R is selected from the group consisting of 3-ethynylphenyl, 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, 3,5-dihydroxyphenyl or phenyl-3-sulfonamide, or a pharmaceutically acceptable salt or a solvate thereof.

In another embodiment, provided herein is a compound of Formula II:

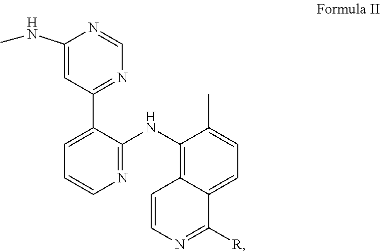

wherein R is NHR¹, wherein R¹ is 2-fluoro-4-iodophenyl, or a pharmaceutically acceptable salt or a solvate thereof.

In another embodiment, provided herein is a compound of Formula III:

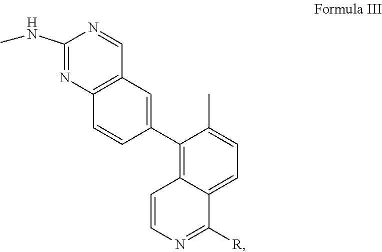

wherein R is NHR¹, wherein R¹ is 3-ethynylphenyl, 3-chloro-4-fluorophenyl, 2-fluoro-4-iodophenyl, or 4-chloro-3-(trifluoromethyl)phenyl, or a pharmaceutically acceptable salt or a solvate thereof.

Ine another embodiment, provided herein is a compound of Formula IV:

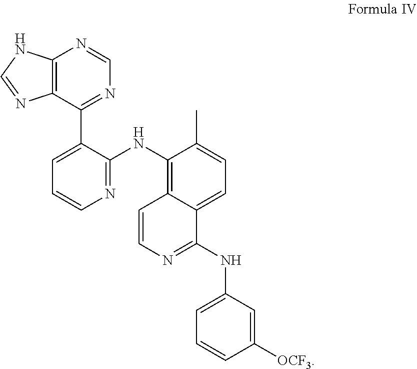 [SMILES: Cc1ccc2c(Nc3cccc(OC(F)(F)F)c3)nccc2c1Nc1ncccc1-c1ncnc2[nH]cnc12]

In one embodiment, the compounds of present disclosure inhibit the activity of BRaf. In one embodiment, the compounds of present disclosure may have an IC50 towards BRaf of about 0.5×10¹⁸ M to about 5×10¹⁸ M, about 1×10¹⁸ M to about 5×10¹⁸ M, about 1×10¹⁸ M to about 3.5×10¹⁸ M, or about 1×10¹⁸ M to about 3×10¹⁸ M.

In one embodiment, the compounds of present disclosure increase the activity of Mitogen-Activated Protein Kinases (MAPK) pathway.

In one embodiment, the activity of MAPK pathway is determined by measuring the phosphorylation of Extracellular Signal-Regulated Kinase (ERK) and calculating a ratio of phospho-ERK to total ERK.

In one embodiment, the activity of MAPK pathway is determined by measuring the paradoxical proliferation of MIA-PaCa-2 cell

In one embodiment, the compounds of the present disclosure increase the ratio of phospho-ERK to total ERK in the MAPK pathway (measurements by at least about 1.025 fold, 1.05 fold, 1.10-fold, 1.15-fold, 1.20-fold, 1.25-fold, 1.30 fold, 1.35-fold, 1.40-fold, 1.45-fold, 1.5-fold, 1.6-fold, 1.7-fold, 1.8-fold, 2-fold, 2.25-fold, 2.5-fold, 2.75-fold, 3-fold, 3.25-fold, 3.5-fold, 3.75-fold, 4-fold, 4.25-fold, 4.5-fold, 4.75-fold, 5-fold, 5.25-fold, 5.50-fold, 5.75-fold, 6-fold, 6.25-fold, 6.50-fold, 6.75-fold, 7-fold, 7.25-fold, 7.5-fold, 8-fold, 8.5-fold, 9-fold, 9.5-fold, 10-fold, 15-fold, 20-fold, 25-fold, 30-fold, 40-fold, 50-fold, 75-fold, 100-fold, 150-fold, or by about 200-fold, including values and ranges therebetween, compared to untreated or control-treated cells.

In one embodiment, the compounds of the present disclosure increase the ratio of phospho-ERK to total ERK by about 1.5-fold to about 50-fold, about 1.5-fold to about 25-fold, 1.5-fold to about 20-fold, about 1.5-fold to about 15-fold, about 2.5-fold to about 15-fold, about 2.5-fold to about 10-fold, about 3-fold to about 20-fold, about 3-fold to about 15-fold, about 4-fold to about 20-fold, about 4-fold to about 15-fold, about 4-fold to about 10-fold, about 5-fold to about 20-fold, about 5-fold to about 15-fold, including values and ranges therebetween, compared to untreated or control-treated cells. In one embodiment, the compounds of the present disclosure increase the level of phospho-ERK relative to total ERK by at least about 2.5%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375% 400%, 425%, 450%, 475%, 500%, 550%, 600%, 650%, 700%, 750%, 800%, 850%, 900%, 950%, 1000%, including values and ranges therebetween, compared to untreated or control-treated cells.

In one embodiment, the compounds of present disclosure show no phototoxicity or low phototoxicity (have a PIF of less than 2). The level of phototoxicity can be determined by measuring a Photo-Irritation Factor (PIF) or a Mean Photo Effect (MPE).

In one embodiment, a PIF can be calculated using the following formula:
PIF=IC50(−Irr)/IC50(+Irr), where PIF>5 indicates phototoxicity; 2<PIF<5 indicates probable phototoxicity; PIF<2 indicates no phototoxicity. In one embodiment, the compounds of present disclosure have a PIF of less than 5. In another embodiment, the compounds of present disclosure have a PIF of less than 2.

In one embodiment, the MPE can be calculated by comparing the complete concentration-response curves. MPE is a weighted average of the difference in response of equivalent doses normalized by the shift in IC50. MPE>0.15 indicates phototoxicity; 0.1<MPE<0.15 indicates probable phototoxicity; MPE<0.1 indicates no phototoxicity. In one embodiment, the compounds of present disclosure have a MPE of less than 0.15. In another embodiment, the compounds of present disclosure have a MPE of less than 0.1.

### Compositions

In one embodiment, provided herein are pharmaceutical compositions comprising a compound of Formula I, II, III, or IV, or a pharmaceutically acceptable salt or a solvate thereof, or a combination thereof, and a pharmaceutically acceptable carrier or excipient.

In one embodiment, provided herein are pharmaceutical compositions comprising a compound of Formula I or a pharmaceutically acceptable salt or a solvate thereof, or a combination thereof, and a pharmaceutically acceptable carrier or excipient.

In one embodiment, the pharmaceutical composition may comprise about 0.01% w/w to about 1% w/w of the compound or a pharmaceutically acceptable salt or a solvate thereof, or a combination thereof, based on the total weight of the composition. In one embodiment, the pharmaceutical composition may comprise about 0.01% w/w to about 0.1% w/w of the compound or a pharmaceutically acceptable salt or a solvate thereof, or a combination thereof, based on the total weight of the composition. For example, the pharmaceutical composition may comprise about 0.01, 0.02, 0.03, 0.04, 0.05, 0.06, 0.07, 0.08, 0.09, 0.1% w/w, including values and ranges therebetween, of any of the compounds disclosed herein or combinations thereof. In one embodiment, the pharmaceutical composition may comprise from about 0.01% to about 0.03%, about 0.01% to about 0.04%, about 0.01% to about 0.05%, about 0.01% to about 0.06%, about 0.01% to 0.07%, about 0.01% to about 0.08%, about 0.01% to about 0.09%, about 0.01% to about 0.1%, 0.02% to about 0.03%, about 0.02% to about 0.04%, about 0.02% to about 0.05%, about 0.02% to about 0.06%, about 0.02% to 0.07%, about 0.02% to about 0.08%, about 0.02% to about 0.02%, about 0.02% to about 0.1%, 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.04% to about 0.05%, about 0.04% to about 0.06%, about 0.04% to 0.07%, about 0.04% to about 0.08%, about 0.04% to about 0.09%, about 0.04% to about 0.1%, %, about 0.05% to about 0.06%, about 0.05% to 0.07%, about 0.05% to about 0.08%, about 0.05% to about 0.09%, about 0.05% to about 0.1% weight/weight (w/w), including values and ranges therebetween, of any of the compounds disclosed herein.

In one embodiment, the pharmaceutical composition may comprise about 0.03% w/w to about 0.25% w/w of the compound or a pharmaceutically acceptable salt or a solvate thereof, or a combination thereof, based on the total weight of the composition. For example, the pharmaceutical composition may comprise about 0.03, 0.04, 0.05, 0.06, 0.07, 0.08, 0.09, 0.1, 0.11, 0.12, 0.13, 01.4, 0.15, 0.16, 0.17, 0.18, 0.19, 0.20, 0.21, 0.22, 0.23, 0.24, 0.25% w/w, including values and ranges therebetween, of any of the compounds disclosed herein or combinations thereof. In one embodiment, the pharmaceutical composition may comprise from about 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.03% to about 0.11%, about 0.03% to about 0.12%, about 0.03% to about 0.13%, about 0.03% to about 0.14%, about 0.03% to about 0.15%, about 0.03% to about 0.16%, about 0.03% to about 0.17%, about 0.03% to about 0.18%, about 0.03% to about 0.19%, about 0.03% to about 0.2%, about 0.03% to about 0.21%, about 0.03% to about 0.22%, about 0.03% to about 0.23%, about 0.03% to about 0.24%, about 0.03% to about 0.25 weight/weight (w/w), including values and ranges therebetween, of any of the compounds disclosed herein.

In one embodiment, the pharmaceutical composition may comprise about 0.01% w/w to about 0.1% w/w of the compound of Formula I or a pharmaceutically acceptable salt or a solvate thereof, or a combination thereof, based on the total weight of the composition. For example, the pharmaceutical composition may comprise about 0.01, 0.02, 0.03, 0.04, 0.05, 0.06, 0.07, 0.08, 0.09, 0.1% w/w, including values and ranges therebetween, of any of the compounds disclosed herein or combinations thereof. In one embodiment, the pharmaceutical composition may comprise from about 0.01% to about 0.03%, about 0.01% to about 0.04%, about 0.01% to about 0.05%, about 0.01% to about 0.06%, about 0.01% to 0.07%, about 0.01% to about 0.08%, about 0.01% to about 0.09%, about 0.01% to about 0.1%, 0.02% to about 0.03%, about 0.02% to about 0.04%, about 0.02% to about 0.05%, about 0.02% to about 0.06%, about 0.02% to 0.07%, about 0.02% to about 0.08%, about 0.02% to about 0.02%, about 0.02% to about 0.1%, 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.04% to about 0.05%, about 0.04% to about 0.06%, about 0.04% to 0.07%, about 0.04% to about 0.08%, about 0.04% to about 0.09%, about 0.04% to about 0.1%, %, about 0.05% to about 0.06%, about 0.05% to 0.07%, about 0.05% to about 0.08%, about 0.05% to about 0.09%, about 0.05% to about 0.1% w/w, including values and ranges therebetween, of any of the compounds disclosed herein.

In one embodiment, the pharmaceutical composition may comprise about 0.03% w/w to about 0.1% w/w of the compound of Formula I or a pharmaceutically acceptable salt or a solvate thereof, or a combination thereof, based on the total weight of the composition. For example, the pharmaceutical composition may comprise about 0.03, 0.04, 0.05, 0.06, 0.07, 0.08, 0.09, 0.1% w/w, including values and ranges therebetween, of any of the compounds disclosed herein or combinations thereof. In one embodiment, the pharmaceutical composition may comprise from about 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.04% to about 0.05%, about 0.04% to about 0.06%, about 0.04% to 0.07%, about 0.04% to about 0.08%, about 0.04% to about 0.09%, about 0.04% to about 0.1%, about 0.05% to about 0.06%, about 0.05% to 0.07%, about 0.05% to about 0.08%, about 0.05% to about 0.09%, about 0.05% to about 0.1% w/w, including values and ranges therebetween, of any of the Formula I-IV compounds disclosed herein.

In one embodiment, the pharmaceutical composition comprising any one of the compounds disclosed herein is formulated for topical administration.

In one embodiment, provided herein is a topical pharmaceutical composition comprising a compound of Formula I, II, III, or IV, or a pharmaceutically acceptable salt or a solvate thereof, or a combination thereof, and a pharmaceutically acceptable carrier or excipient.

Compositions for topical administration (topical compositions) can be formulated in the form of a gel, a hydrogel, an ointment, a cream, a paste, a foam, a spray, a lotion, a liquid, or a dermal patch and may comprise any of the disclosed compound(s) in any of the amounts described herein. In one embodiment, provided herein is a topical pharmaceutical composition comprising a compound of Formula I or a pharmaceutically acceptable salt or a solvate thereof, or a combination thereof, and a pharmaceutically acceptable carrier or excipient. Compositions for topical administration (topical compositions) can be in the form of a gel, a hydrogel, an ointment, a cream, a paste, a foam, a spray, a lotion, a liquid, or a dermal patch and may comprise any of the disclosed compound(s) in any of the amounts described herein.

In one embodiment, the topical composition is a gel and comprises about 0.01% w/w to about 1% w/w of the compound of Formula I or a pharmaceutically acceptable salt or a solvate thereof, or a combination thereof, and a pharmaceutically acceptable carrier or excipient.

In one embodiment, the topical composition is a gel and comprises about 0.03% w/w to about 0.25% w/w of the compound of Formula I or a pharmaceutically acceptable salt or a solvate thereof, or a combination thereof, and a pharmaceutically acceptable carrier or excipient.

In one embodiment, the topical composition is a gel and comprises about 0.03% w/w to about 0.1% w/w of the compound of Formula I or a pharmaceutically acceptable salt or a solvate thereof, or a combination thereof, and a pharmaceutically acceptable carrier or excipient.

In one embodiment, the topical composition is a gel and comprises about 0.01% w/w to about 0.1% w/w of the compound of Formula I or a pharmaceutically acceptable salt or a solvate thereof, or a combination thereof, and a pharmaceutically acceptable carrier or excipient.

In one embodiment, the topical composition is a gel and comprises about 0.03% w/w of the compound of Formula I or a pharmaceutically acceptable salt or a solvate thereof, or a combination thereof, and a pharmaceutically acceptable carrier or excipient.

Topical compositions useful in the present disclosure may be formulated as a solution. Such compositions may comprise an emollient preferably containing from about 1% to about 50% of an emollient(s). As used herein, the term “emollient” refers to materials used for the prevention or relief of dryness, as well as for the protection of the skin. A number of suitable emollients are known and may be used in the present disclosure. For example, Sagarin, Cosmetics, Science and Technology, 2nd Edition, Vol. 1, pp. 32-43 (1972) and the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”) contains numerous examples of suitable materials.

A lotion can be made from such a solution. Lotions typically comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may comprise a simple base of animal or vegetable oils or semisolid hydrocarbons. An ointment may comprise from about 2% to about 10% of an emollient(s) plus from about 0.1% to about 2% of a thickening agent(s). A more complete disclosure of thickening agents or viscosity increasing agents useful herein can be found in Sagarin, Cosmetics, Science and Technology, 2nd Edition, Vol. 1, pp. 72-73 (1972) and the ICI Handbook pp. 1693-1697.

The topical compositions useful in the present disclosure may be formulated as emulsions. If the carrier for a topical composition is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, in McCutcheon's Detergents and Emulsifiers, North American Edition, pp. 317-324 (1986), and the ICI Handbook, pp. 1673-1686.

Lotions and creams can be formulated as emulsions. Such lotions may comprise from 0.5% to about 5% of an emulsifier(s). Creams may comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

The topical compositions of this disclosure can also be formulated as a gel (e.g., an aqueous, alcohol, alcohol/water, or oil gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils include, but are not limited to, hydrogenated butylene/ethylene/styrene copolymer and hydrogenated ethylene/propylene/styrene copolymer. Gel compositions may comprise between about 0.1% and 5%, by weight, of such gelling agents.

Another topical dosage form is a paste. Pastes are stiff semi-solid topical dosage forms, containing a high proportion of finely powdered solid such as starch, zinc oxide, calcium carbonate and talc. Another component of pastes is a base, which may be a hydrocarbon, an absorption ingredient, a water-miscible ingredient or a water-soluble ingredient. Pastes are less greasy than ointments.

In one embodiment, the topical compositions of this disclosure can also be formulated as a spray able topical formulation. The compositions can be in a form suitable for application by spraying from an aerosol or pump spray container. The spray formulation can comprise the compound of the disclosure a solution or suspension in a vehicle. The vehicle can optionally contain a polymer or combination of polymers which, when sprayed on the surface of the skin, forms a film on the skin.

In addition to the above carriers and excipients, other emollients and surface active agents can be incorporated into the topical compositions, including glycerol trioleate, acetylated sucrose distearate, sorbitan trioleate, polyoxyethylene (1) monostearate, glycerol monooleate, sucrose distearate, polyethylene glycol (50) monostearate, octylphenoxypoly (ethyleneoxy) ethanol, decaglycerin penta-isostearate, sorbitan sesquioleate, hydroxylated lanolin, lanolin, triglyceryl diisostearate, polyoxyethylene (2) oleyl ether, calcium stearoyl-2-lactylate, methyl glucoside sesquistearate, sorbitan monopalmitate, methoxy polyethylene glycol-22/dodecyl glycol copolymer (Elfacos E200), polyethylene glycol-45/dodecyl glycol copolymer (Elfacos ST9), polyethylene glycol 400 distearate, and lanolin derived sterol extracts, glycol stearate and glycerol stearate; alcohols, such as cetyl alcohol and lanolin alcohol; myristates, such as isopropyl myristate; cetyl palmitate; cholesterol; stearic acid; propylene glycol; glycerin, sorbitol and the like.

The compounds or compositions described herein can be used alone or in combination with other active agents. In one embodiment, the compounds or compositions for the treatment or prevention of a cutaneous adverse reaction of EGFR inhibitors, PI3K inhibitors, MEK inhibitors, RAS inhibitors, ERK inhibitor, SHP inhibitor, HER dimerization inhibitors or combinations thereof are topically administered in a subject in need thereof without coadministration of another active agent. In one embodiment, the active agent of the composition provided herein consists of a compound having Formula I, II, III, or IV.

### Methods

Provided herein are methods of treating, preventing, and/or ameliorating the dermatological or cutaneous adverse reactions induced by chemotherapy or biologic agents inhibiting members of the MAPK pathway downstream to EGFR, such as EGFR inhibitors, RAS inhibitors, ERK inhibitors, PI3K inhibitors, SHP inhibitors, MEK inhibitors, HER inhibitors or combinations thereof.

Among the various signaling cascades, RAS-RAF-MEK-ERK is the most important pathway (Development of small molecule extracellular signal-regulated kinases (ERKs) inhibitors for cancer therapy, Xiaoli Pan ^{a †}, J et al., Acta Pharmaceutica Sinica B, Vol. 1, Issue 5, May 2022, p. 2171-2192).

A. A. Samatar, P. I. Poulikakos. Targeting RAS-ERK signaling in cancer: promises and challenges Nat Rev Drug Discov, 13 (2014), pp. 928-942U. Degirmenci, M. Wang, J. Hu. Targeting aberrant RAS/RAF/MEK/ERK signaling for cancer therapy Cells, 9 (2020), p. 198.

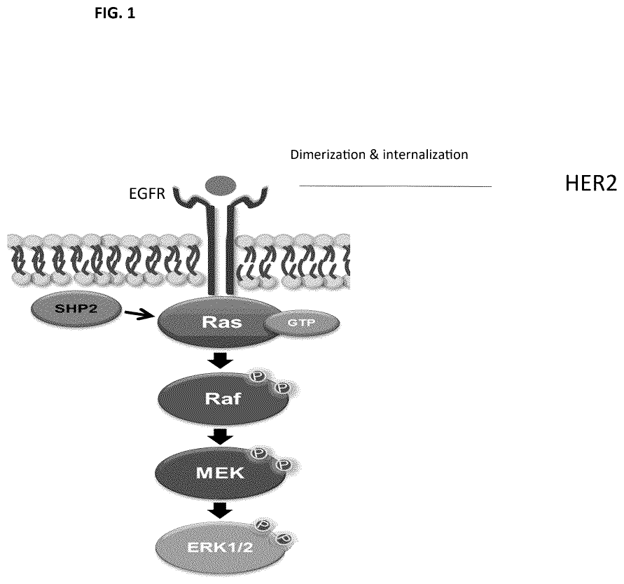

See FIG. 1.

In one embodiment, provided herein is a method for treating, ameliorating, and/or preventing a cutaneous adverse reaction of EGFR inhibitors RAS inhibitors, SHP inhibitors, ERK inhibitors, PI3K inhibitors, MEK inhibitors, HER dimerization inhibitors or combinations thereof in a subject in need thereof, comprising administering a therapeutically effective amount of a composition comprising a compound of Formula I, Formula II, Formula III, Formula IV, or a combination thereof; and a pharmaceutically acceptable carrier or excipient.

The adverse events caused by the treatment of a cancer subject in need thereof not only with EGFR inhibitors, PI3K inhibitors, MEK inhibitors, and/or HER inhibitors but also the adverse effects caused by RAS inhibitors or ERK inhibitors or SHP inhibitors can impair the patient's quality of life and adherence to treatment and in severe cases may require dose reduction or even temporary/permanent interruption of therapy (Boone S L, Rademaker A, Liu D, et al. Impact and management of skin toxicity associated with anti-epidermal growth factor receptor therapy: survey results. Oncology. 2007; 72:152).

It occurred to the inventor of the instant disclosure, that the compounds of Formula I-IV may be effective for prevention, amelioration or treatment of cutaneous adverse effects not only at the level of EGFR, but also at the level of RAS and/or ERK,

In one embodiment, provided herein is a method for treating, ameliorating, and/or preventing EGFR inhibitors, RAS inhibitors, SHP inhibitors, PI3K inhibitors, MEK inhibitors, and/or HER dimerization inhibitors-induced acneiform lesions in a subject in need thereof, comprising administering a therapeutically effective amount of a composition comprising a compound of Formula I, Formula II, Formula III, Formula IV, or a combination thereof; and a pharmaceutically acceptable carrier or excipient.

In one embodiment, provided herein is a method for treating, ameliorating, and/or preventing a cutaneous adverse reaction of EGFR inhibitors, RAS inhibitors, ERK inhibitors, SHP inhibitors, PI3K inhibitors, MEK inhibitors, HER dimerization inhibitors or combinations thereof in a subject in need thereof, comprising administering a therapeutically effective amount of a composition comprising a compound of Formula I; and a pharmaceutically acceptable carrier or excipient.

In one embodiment, provided herein is a method for treating, ameliorating, and/or preventing EGFR inhibitors, RAS inhibitors, ERK inhibitors, SHP inhibitors, PI3K inhibitors, MEK inhibitors, and/or HER dimerization inhibitors-induced acneiform lesions in a subject in need thereof, comprising administering a therapeutically effective amount of a composition comprising a compound of Formula I; and a pharmaceutically acceptable carrier or excipient.

In one embodiment, methods for treating, ameliorating, and/or preventing a cutaneous adverse reaction of EGFR inhibitors, PI3K inhibitors, RAS inhibitors, ERK inhibitors, SHP inhibitors, MEK inhibitors, HER inhibitors or combinations thereof in a subject in need thereof comprise administering topically a therapeutically effective amount of a topical pharmaceutical composition comprising a compound of Formula I, Formula II, Formula III, Formula IV, or a combination thereof, or a pharmaceutically acceptable salt or a solvate thereof, or a combination thereof; and a pharmaceutically acceptable carrier or excipient

It also occurred to the inventor of the present disclosure that besides the prevention, amelioration and treatment of the cutaneous lesions, the administration of the compositions comprising compounds of Formula I-IV has a no less important effect on the subject in need thereof: as the cutaneous lesions become less of a bother, the adherence (patient compliance) of the subjects to the anti-cancer treatment is greatly improved.

In one embodiment, methods for treating, ameliorating, and/or preventing a cutaneous adverse reaction of EGFR inhibitors, RAS inhibitors, ERK inhibitors, SHP inhibitors, PI3K inhibitors, MEK inhibitors, HER dimerization inhibitors or combinations thereof in a subject in need thereof comprise administering topically a therapeutically effective amount of a topical pharmaceutical composition comprising a BRaf inhibitor compound of Formula I, or a pharmaceutically acceptable salt or a solvate thereof, or a combination thereof; and a pharmaceutically acceptable carrier or excipient.

In one embodiment, methods for treating, ameliorating, and/or preventing a cutaneous adverse reaction of EGFR inhibitors, PI3K inhibitors, MEK inhibitors, ERK inhibitors, SHP inhibitors, RAS inhibitor, HER dimerization inhibitors or combinations thereof in a subject in need thereof comprise administering a therapeutically effective amount of a composition comprising a combination of any of the compounds disclosed herein and a pharmaceutically acceptable carrier or excipient.

In one embodiment, methods for treating, ameliorating, and/or preventing a cutaneous adverse reaction of EGFR inhibitors, PI3K inhibitors, MEK inhibitors, ERK inhibitors, SHP inhibitors, RAS inhibitors, HER dimerization inhibitors or combinations thereof in a subject in need thereof comprise administering a therapeutically effective amount of a composition comprising a compound of formula I in any of the w/w % amounts disclosed herein and a pharmaceutically acceptable carrier or excipient.

Dermatological or cutaneous adverse reactions induced by anti-cancer treatment agents such as EGFR inhibitors, RAS inhibitors or combinations thereof include acneiform rash, acneiform lesions, papulopustular rash, abnormal scalp hair growth, abnormal facial hair growth, abnormal hair growth, abnormal eyelash growth, xerosis, pruritus, paronychia with or without pyogenic granulomas and telangiectasia.

The methods described herein treat, ameliorate, and/or prevent one or more of these adverse reactions.

In some embodiment, a cutaneous adverse reaction of a RAS inhibitor, or combinations thereof that is treated, ameliorated, and/or prevented by the compounds/compositions of the present disclosure is an acneiform lesion (also referred herein as acneiform rash).

In some embodiment, a cutaneous adverse reaction of an ERK inhibitor, or combinations thereof that is treated, ameliorated, and/or prevented by the compounds/compositions of the present disclosure is an acneiform lesion (also referred herein as acneiform rash).

In some embodiment, a cutaneous adverse reaction of an SHP inhibitor, or combinations thereof that is treated, ameliorated, and/or prevented by the compounds/compositions of the present disclosure is an acneiform lesion (also referred herein as acneiform rash).

In one embodiment, the subject is a mammal, human or non-human. In one embodiment, the subject is a human.

In one embodiment, the subject is receiving a RAS inhibitor or a combination thereof at the time of the administration of the compounds/compositions of the present disclosure. In one embodiment, the subject has solid tumor, melanoma, lung cancer, head & neck cancer, colorectal cancer, brain cancer, multiple myeloma, breast cancer, pancreatic cancer, liver cancer and neurofibromatosis.

In one embodiment, the subject is a mammal, human or non-human. In one embodiment, the subject is a human.

In one embodiment, the subject is receiving a ERK inhibitor or a combination thereof at the time of the administration of the compounds/compositions of the present disclosure. In one embodiment, the subject has solid tumor, melanoma, lung cancer, head & neck cancer, colorectal cancer, brain cancer, multiple myeloma, breast cancer, pancreatic cancer, liver cancer and neurofibromatosis.

In one embodiment, the subject is a mammal, human or non-human. In one embodiment, the subject is a human.

In one embodiment, the subject is receiving a SHP inhibitor or a combination thereof at the time of the administration of the compounds/compositions of the present disclosure. In one embodiment, the subject has solid tumor, melanoma, lung cancer, head & neck cancer, colorectal cancer, brain cancer, multiple myeloma, breast cancer, pancreatic cancer, liver cancer and neurofibromatosis.

In another embodiment, the compounds/compositions of the present disclosure are administered to the subject prior to or after administration of an EGFR inhibitor, PI3K inhibitor, MEK inhibitor, RAS inhibitor, ERK inhibitor, SHP inhibitor, HER dimerization inhibitors or a combination thereof.

In another embodiment, the compounds/compositions of the present disclosure are administered to the subject prior to or after appearance of the cutaneous reactions.

In one embodiment, methods disclosed herein comprise topical administration of a therapeutically effective amount of the compounds/compositions of the present disclosure.

Methods comprising topical administration comprise local administration or application of any of the compositions disclosed herein to the skin of the subject. In one embodiment, topical administration comprises topically administering a composition, wherein the composition is a gel, a hydrogel, an ointment, a cream, a spray, a dermal patch, a paste, a foam, a lotion or a liquid. In one embodiment, the amount of the compound administered depends on the nature of the compound, the formulation, and/or the severity of the cutaneous reaction. The therapeutically effective amount that is administered to a patient can be determined by dose-ranging clinical studies known in the art.

In one embodiment, the dosage regimen and/or length of treatment depends on the nature of the compound, the formulation, and/or the severity of the cutaneous reaction.

According to one embodiment, the method comprises topically administering, once daily, to a skin area affected by the EGFR inhibitors, PI3K inhibitors, RAS inhibitor, ERK inhibitor, SHP inhibitor, MEK and/or HER dimerization-inhibitors induced of a subject in need thereof the topical pharmaceutical composition. In one embodiment, the method comprises topically administering, once daily, to a skin area affected by the EGFR inhibitors-induced cutaneous lesions the topical pharmaceutical composition.

In one embodiment, the subject in need thereof is treated with EGFR inhibitors. In one embodiment, the subject in need thereof is treated with PI3K inhibitors. In one embodiment, the subject in need thereof is treated with MEK inhibitors. In one embodiment, the subject in need thereof is treated with EGFR inhibitors, PI3K inhibitors, MEK inhibitors, RAS inhibitor, ERK inhibitor, SHP inhibitor, and/or HER dimerization inhibitors. In some embodiment, a subject having cancer is treated with EGFR inhibitors.

In one embodiment, the subject in need thereof has grade 1 acneiform lesions. In one embodiment, the subject in need thereof has grade 2 acneiform lesions. In one embodiment, the subject in need thereof has grade 3 acneiform lesions. In one embodiment, the subject in need thereof has grade 4 acneiform lesions.

In one embodiment, the topical pharmaceutical composition is administered once daily for 2 weeks, 3 weeks, 4 weeks, 5 weeks, 6 weeks, 7 weeks, 8 weeks, 9 weeks, 10 weeks, 11 weeks, 12 weeks, etc. In one embodiment, the topical composition is administered once daily for 4 to 12 weeks.

In one embodiment, the topical pharmaceutical composition is administered twice daily for 2 weeks, 3 weeks, 4 weeks, 5 weeks, 6 weeks, 7 weeks, 8 weeks, 9 weeks, 10 weeks, 11 weeks, 12 weeks, etc. In one embodiment, the topical pharmaceutical composition is administered twice daily for 4 to 12 weeks.

In one embodiment, the method comprises topically administering, once daily, to a skin area affected by the cutaneous lesions the topical pharmaceutical composition comprising from about 0.01% to 1% w/w, from 0.01% to 0.1% w/w, from 0.01% to 0.3% w/w, from 0.03% to 1% w/w, from 0.03% to 0.3% w/w, from 0.03% to 0.1% w/w, of the compound of Formula I or a pharmaceutically acceptable salt or a solvate thereof, or a combination thereof, and a pharmaceutically acceptable carrier or excipient. In one embodiment, the topical pharmaceutical composition is administered once daily for 4 to 12 weeks.

In one embodiment, the method comprises topically administering, twice daily, to a skin area affected by the cutaneous lesions the topical pharmaceutical composition comprising from about 0.01% to 1% w/w, from 0.01% to 0.1% w/w, from 0.01% to 0.3% w/w, from 0.03% to 1% w/w, from 0.03% to 0.3% w/w, from 0.03% to 0.1% w/w of the compound of Formula I or a pharmaceutically acceptable salt or a solvate thereof, or a combination thereof, and a pharmaceutically acceptable carrier or excipient. In one embodiment, the topical pharmaceutical composition is administered twice daily for 4 to 12 weeks.

In one embodiment, the methods reduce the severity of the cutaneous adverse reactions, for example as using the NCI CTCAE version 5.0 skin and subcutaneous tissue disorders grading scale. For example, the methods reduce the severity of the cutaneous adverse lesions from grade 2 to grade 1. In one embodiment, the methods prevent the escalation of the acneiform lesions. In one embodiment, the methods reduces the severity of the acneiform lesions from grade 4 to grade 3, 2, 1, or 0, the method reduces the severity of the acneiform lesions from grade 3 to grade 2, 1, or 0, the method reduces the severity of the acneiform lesions from grade 3 to grade 2, 1, or 0, the method reduces the severity of the acneiform lesions from grade 2 to grade 1, or 0, the method reduces the severity of the acneiform lesions from grade 1 to grade 0.

In one embodiment, the administration results in a significant reduction in severity of the cutaneous adverse reaction as early as 1 week after the initial administration of the topical pharmaceutical composition. In one embodiment, the methods reduce the severity of the cutaneous adverse reactions, for example as using the NCI CTCAE version 5.0 skin and subcutaneous tissue disorders grading scale. In one embodiment, the methods reduce the severity of the cutaneous adverse lesions from grade 2 to grade 1.

In one embodiment, the method provides sustained benefit for at least 7 days, 14 days, 21 days 28 days, 35 days, 42 days, 49 days, 56 days, or more after administration had terminated.

In one embodiment, the method prevents the apparition of EGFR inhibitors, PI3K inhibitors, MEK inhibitors, RAS inhibitor, ERK inhibitor, SHP inhibitor, and/or HER dimerization inhibitors-induced cutaneous lesions when administered concomitantly with the EGFR inhibitors, PI3K inhibitors, RAS inhibitor, ERK inhibitor, SHP inhibitor, MEK-inhibitors and/or HER dimerization inhibitors.

In one embodiment, the methods disclosed herein reduce the severity of the cutaneous adverse reactions.

Success of treating cutaneous adverse reactions can be measured using methods known in the art.

The most validated, commonly used and FDA approved system to grade the severity of cutaneous adverse reactions is National Cancer Institute's Common Terminology Criteria for Adverse Events (CTCAE) version 5.0, which recognizes 6 grades shown in Table 1 below. (with the additional of grade 0 for healthy subject who does not present any level of rash).

TABLE 1
NCI-CTCAE version 5.0 grading scale of skin and subcutaneous tissue disorders

| | |
|---|---|
| Grade 1 | Papules and/or pustules covering <10% of the BSA which may or |
| | may not be associated with symptoms of pruritus or tenderness |
| Grade 2 | Papules and/or pustules covering 10-30% of the BSA which may or may |
| | not be associated with symptoms of pruritus or tenderness; associated |
| | with psychosocial impact; limiting instrumental ADL; papules and/or |
| | pustules covering >30% BSA with or without mild symptoms |
| Grade 3 | Papules and/or pustules covering >30% BSA with moderate or severe |
| | symptoms; limiting self-care ADL, associated with local superinfection |
| | with oral antibiotics indicated |
| Grade 4 | Life-threatening consequences; papules and/or pustules covering any % |
| | BSA, which may or may not be associated with symptoms of pruritus |
| | or tenderness and are associated with extensive superinfection with IV |
| | antibiotics indicated |

Grade 5- Death
BSA = Body surface area; ADL = activity of daily living

In one embodiment, the methods disclosed herein reduce the severity of the cutaneous adverse reactions from grade 4 to grade 3, 2, 1, or 0, as defined by National Cancer Institute Common Terminology Criteria for Adverse Events (NCI-CTCAE) version 5.0.

In one embodiment, the methods disclosed herein reduce the severity of the cutaneous adverse reactions from grade 3 to grade 2, 1, or 0, as defined by NCI-CTCAE version 5.0.

In one embodiment, the methods disclosed herein reduce the severity of the cutaneous adverse reactions from grade 2 to grade 1 or 0, as defined by NCI-CTCAE version 5.0.

In one embodiment, the methods disclosed herein reduce the severity of the cutaneous adverse reactions from grade 1 to grade 0, as defined by NCI-CTCAE version 5.0.

In one embodiment, the methods disclosed herein prevent, partially or completely, the development of cutaneous adverse reactions.

In one embodiment, the methods disclosed herein prevent, partially or completely, the development of grade 4, grade 3, grade 2, or grade 1 of the cutaneous adverse reactions, as defined by NCI-CTCAE version 5.0.

In one embodiment, the methods disclosed herein prevent the escalation of the cutaneous adverse reaction. For example, in one embodiment, the methods disclosed herein prevent the escalation of the cutaneous adverse reaction from grade 0 to grade 1, 2, 3, or 4, as defined by NCI-CTCAE version 5.0. In another embodiment, the methods disclosed herein prevent the escalation of the cutaneous adverse reaction from grade 1 to grade 2, 3, or 4, as defined by NCI-CTCAE version 5.0. In another embodiment, the methods disclosed herein prevent the escalation of the cutaneous adverse reaction from grade 2 to grade 3 or 4, as defined by NCI-CTCAE version 5.0. In another embodiment, the methods disclosed herein prevent the escalation of the cutaneous adverse reaction from grade 3 to grade 4, as defined by NCI-CTCAE version 5.0.

Another system that may be used to grade the severity of cutaneous adverse reactions is Lacouture grading scale shown in Table 2 below.

TABLE 2

| Adverse Event | Grade 1 | Grade 2 | Grade 3 | Grade 4 |
|---|---|---|---|---|
| Papulopustular | 1A. Papules or | 2A. Papules or pustules 6- | 3A. Papules or | |
| eruption | pustules <5; | 20; | pustules >20; OR | |
| (Grading | OR 1 area of | OR 2-5 areas of erythema | more than 5 areas | |
| individually for | erythema or | or edema <1 cm in size | of erythema or | |
| face, scalp, chest | edema <1 cm in | | edema <1 cm in | |
| or back) | size | | size | |
| | 1B. Papules or | 2B. Papules or pustules 6- | 3B. Papules or | |
| | pustules <5; | 20; OR 2-5 areas of | pustules >20; OR | |
| | OR 1 area of | erythema or edema <1 cm | more than 5 areas | |
| | erythema or | in size AND pain, | of erythema or | |
| | edema <1 cm in | pruritus, or effect on | edema <1 cm in | |
| | size AND pain | emotions or functioning | size; AND pain, | |
| | or pruritus | | pruritus, or effect | |
| | | | on emotions or | |
| | | | functioning | |
| Nail changes | Onycholysis or | Onycholysis with | Nail plate changes | |
| Nail Plate | ridging without | mild/moderate pain; any | interfering with | |
| | pain | nail plate lesion | self-care ADL | |
| | | interfering with | | |
| | | instrumental ADL | | |
| Nail changes | Disruption or | Erythematous/tender/painful; | Periungual | |
| Nail fold | absence of | OR pyogenic | abscess: OR fold | |
| | cuticle; OR | granuloma; OR crusted | changes interfering | |
| | erythema | lesions: OR any fold | with self-care | |
| | | lesion interfering with | ADL | |
| | | instrumental AD | | |
| Nail changes | Xerosis | Xerosis AND/OR | Digit tip lesions | |
| Digit tip | AND/OR | erythema with | interfering with | |
| | erythema | mild/moderate pain or | self-care ADL | |
| | without pain | stinging; OR fingertip | | |
| | | fissures; OR any digit tip | | |
| | | lesion interfering with | | |
| | | instrumental ADL | | |
| Erythema | Painless | Painful erythema, | Painful erythema, | |
| | erythema, | blanching; erythema | nonblanching; | |
| | blanching; | covering 10-30% BSA | erythema | |
| | erythema | | covering >30% | |
| | covering <10% | | BSA | |
| | BSA | | | |
| Pruritus | Mild OR | 2A. Moderate localized | Severe, | |
| | localized, | OR widespread | widespread | |
| | intermittent, not | intermittent AND | constant AND | |
| | requiring | requiring intervention | interfering with | |
| | therapy. | 2B. Moderate localized | sleep | |
| | | OR widespread constant | | |
| | | AND requiring | | |
| | | intervention | | |
| Xerosis | Scaling/flaking | 2A. Scaling/flaking | 3A. | |
| | covering <10% | covering 10-30% BSA + | Scaling/flaking | |
| | BSA NO | pruritus OR effect on | covering >30% | |
| | erythema/pruritus/ | emotions/functioning | BSA AND pruritus | |
| | effect on | | AND erythema | |
| | emotions or | | AND effect on | |
| | functioning | | emotions/functioning | |
| | | | AND + | |
| | | | fissuring/cracking | |
| | | 2B. Scaling/flaking + | 3B. | |
| | | pruritus Covering 10-30% | Scaling/flaking | |
| | | BSA AND effect on | covering >30% | |
| | | emotions/functioning + | BSA AND pruritus | |
| | | erythema | AND erythema | |
| | | | AND effect on | |
| | | | emotions/ | |
| | | | functioning AND | |
| | | | fissuring/cracking + | |
| | | | signs of super | |
| | | | infection | |
| Hair changes: | Terminal hair | 2A. Hair loss associated | | |
| Scalp hair loss | loss <50% of | with marked increase in | | |
| or alopecia | normal for that | shedding and 50%-74% | | |
| | individual that | loss compared to normal | | |
| | may or may not | for that individual. Hair | | |
| | be noticeable to | loss is apparent to others, | | |
| | others but is | may be difficult to | | |
| | associated with | camouflage with change | | |
| | increased | in hair style and may | | |
| | shedding and | require hairpiece. | | |
| | overall feeling | 2B. Marked loss of at | | |
| | of less volume. | least 75% hair compared | | |
| | May require | to normal for that | | |
| | different hair | individual with inability | | |
| | style to cover | to camouflage except with | | |
| | but does not | a full wig OR new | | |
| | require | cicatricial hair loss | | |
| | hairpiece to | documented by biopsy | | |
| | camouflage | that covers at least 5% | | |
| | | scalp surface area. May | | |
| | | impact on functioning in | | |
| | | social, personal or | | |
| | | professional situations. | | |
| Hair Changes: | Some distortion | 2A. Distortion of hair | | |
| disruption of | of hair growth | growth in many hairs in a | | |
| normal hair | but does not | given area that cause | | |
| growth | cause symptoms | discomfort or symptoms | | |
| (specify): | or require | that may require | | |
| Facial hair | intervention. | individual hairs to be | | |
| (diffuse, not just in | | removed. | | |
| male | | 2B. Distortion of hair | | |
| beard/mustache | | growth of most hairs in a | | |
| areas) | | given area with symptoms | | |
| Eyelashes | | or resultant problems | | |
| Eyebrows | | requiring removal of | | |
| Body Hair | | multiple hairs. | | |
| Beard and | | | | |
| moustache hair | | | | |
| (hirsutism) | | | | |
| Hair Changes: | Increase in | 2A. Increase in length, | | |
| increased | length, | thickness and/or density | | |
| hair growth | thickness and/or | of hairs that is very | | |
| (specify): | density of hair | noticeable and requires | | |
| Facial hair | that the patient | regular shaving or | | |
| (diffuse, not just in | is able to | removal of hairs in order | | |
| male | camouflage by | to camouflage. May cause | | |
| beard/mustache | periodic | mild symptoms related to | | |
| areas) | shaving, | hair overgrowth. | | |
| Eyelashes | bleaching or | 2B. Marked increase in | | |
| Eyebrows | removal of | hair density, thickness | | |
| Body Hair | individual hairs. | and/or length of hair that | | |
| Beard and | | requires either frequent | | |
| moustache | | shaving or destruction of | | |
| hair (hirsutism) | | the hair to camouflage. | | |
| | | May cause symptoms | | |
| | | related to hair | | |
| | | overgrowth. Without hair | | |
| | | removal, inability to | | |
| | | function normally in | | |
| | | social, personal or | | |
| | | professional situations. | | |
| Flushing | 1A. Face OR | 2A. Symptomatic on face, | 3A. Face and | |
| | chest, | or chest, transient | chest, transient, | |
| | asymptomatic, | | symptomatic | |
| | transient | | | |
| | 1B. Any | 2B. Symptomatic on face, | 3B. Face and | |
| | location, | or chest permanent | chest, permanent, | |
| | asymptomatic, | | symptomatic | |
| | permanent | | | |
| Telangiectasia | One area (<1 cm | 2A. 2-5 (1 cm diameter) | More than 6 (1 cm | |
| | diameter) NOT | areas NOT affecting | diameter) OR | |
| | affecting | emotions or functioning | confluent areas | |
| | emotions or | 2B. 2-5 (1 cm diameter) | affecting emotions | |
| | functioning | areas affecting emotions | or functioning | |
| | | or functioning | | |
| Hyperpigmentation | One area (<1 cm | 2A. 2-5 (1 cm diameter) | More than 6 (1 cm | |
| | diameter) NOT | areas NOT affecting | diameter) OR | |
| | affecting | emotions or functioning | confluent areas | |
| | emotions or | 2B. 2-5 (1 cm diameter) | affecting emotions | |
| | functioning | areas affecting emotions | or functioning | |
| | | or functioning | | |
| Mucositis | Mild erythema | Symptomatic (mild pain, | Pain requiring | Erythema |
| Oral | or edema, and | opiod not required): | opiod analgesic; | and |
| Anal | asymptomatic | erythema or limited | erythema and | ulceration, |
| | | ulceration, can eat solid | ulceration, cannot | cannot |
| | | foods and take oral | eat solids, can | tolerate PO |
| | | medication (Oral | swallow liquids | intake; |
| | | mucositis only) | (Oral mucositis | require tube |
| | | | only) | feeding or |
| | | | | hospitalization |
| | | | | (Oral |
| | | | | mucositis |
| | | | | only) |
| Radiation | Faint erythema | Moderate to brisk | Moist | Skin |
| dermatitis | or dry | erythema; patchy moist | desquamation | necrosis or |
| | desquamation | desquamation, mostly | other than skin | ulceration of |
| | | confined to skin folds and | folds and creases; | full |
| | | creases; moderate edema | bleeding induced | thickness |
| | | | by minor trauma or | dermis; |
| | | | abrasion | spontaneous |
| | | | | bleeding |
| | | | | from |
| | | | | involved site |
| Hyposalivation | Can eat but | Moderate/thickened | No saliva, unable | |
| | requires liquids, | saliva: cannot eat dry | to speak without | |
| | no effect on | foods, mild speech | water, no oral | |
| | speech | impairment (sticky | intake without | |
| | | tongue, lips, affecting | water | |
| | | speech) | | |
| Taste | Altered or | Altered or reduced taste | Taste | |
| | reduced taste; | affecting interest and | abnormalities, | |
| | no impact on | ability to eat no | requires | |
| | oral intake | intervention required | intervention | |

In one embodiment, the methods disclosed herein reduce the severity of the Cutaneous adverse reactions from grade 4 to grade 3B, 3A, 2B, 2A, IB, or 1A, as defined by Lacouture grading scale.

In one embodiment, the methods disclosed herein reduce the severity of the cutaneous adverse reactions from grade 3B to grade 3A, 2B, 2A, IB, or 1A, as defined by Lacouture grading scale.

In one embodiment, the methods disclosed herein reduce the severity of the cutaneous adverse reactions from grade 3A to grade 2B, 2A, IB, or 1A, as defined by Lacouture grading scale.

In one embodiment, the methods disclosed herein reduce the severity of the cutaneous adverse reactions from grade 2B to grade 2A, IB, or 1A, as defined by Lacouture grading scale.

In one embodiment, the methods disclosed herein reduce the severity of the cutaneous adverse reactions from grade 2A to grade IB or 1 A, as defined by Lacouture grading scale.

In one embodiment, the methods disclosed herein reduce the severity of the cutaneous adverse reactions from grade IB to grade 1A, as defined by Lacouture grading scale.

In one embodiment, the methods disclosed herein prevent, partially or completely, the development of grade 4, grade 3B, grade 3A, grade 2B, grade 2A, grade IB, or grade 1A of the cutaneous adverse reactions, as defined by Lacouture grading scale.

In one embodiment, the methods disclosed herein prevent the escalation of the cutaneous adverse reaction from grade 1 A to grade IB, 2 A, 2B, 3 A, 3B or 4, as defined by Lacouture grading scale. In another embodiment, the methods disclosed herein prevent the escalation of the cutaneous adverse reaction from grade IB to grade 2A, 2B, 3A, 3B or 4, as defined by Lacouture grading scale. In one embodiment, the methods disclosed herein prevent the escalation of the cutaneous adverse reaction from grade 2A to grade 2B, 3A, 3B or 4, as defined by Lacouture grading scale. In one embodiment, the methods disclosed herein prevent the escalation of the cutaneous adverse reaction from grade 2B to grade 3A, 3B or 4, as defined by Lacouture grading scale. In one embodiment, the methods disclosed herein prevent the escalation of the cutaneous adverse reaction from grade 3A to grade 3B or 4, as defined by Lacouture grading scale. In one embodiment, the methods disclosed herein prevent the escalation of the cutaneous adverse reaction from grade 3B to grade 4, as defined by Lacouture grading scale.

In one embodiment, a method of treating and/or preventing EGFR inhibitor, PI3K inhibitor, RAS inhibitor, ERK inhibitor, SHP inhibitor, MEK inhibitor and/or HER dimerization inhibitor-induced acneiform lesions in a subject in need thereof is provided, the method comprising administering once a day for 4 to 6 weeks to the skin of the subject a topical pharmaceutical composition comprising from 0.01% weight/weight to 0.3% weight/weight of a compound of Formula I and a pharmaceutically acceptable carrier or excipient to the subject for treating EGFR inhibitor, PI3K inhibitor, RAS inhibitor, ERK inhibitor, SHP inhibitor, MEK inhibitor and/or HER dimerization inhibitor-induced acneiform lesions. In one embodiment, the topical pharmaceutical composition is applied for four to twelve weeks to the skin of the subject in need thereof.

In one embodiment, the topical composition comprises from 0.03% to 0.25% weight/weight of the compound of Formula I. In one embodiment, the topical composition comprises about 0.03% weight/weight of the compound of Formula I.

In one embodiment, the topical pharmaceutical composition is in the form of a gel, a hydrogel, an ointment, a cream, a spray, a dermal patch, a foam, a lotion or a liquid. In one embodiment, the topical pharmaceutical composition is in the form of a gel. In one embodiment, the topical pharmaceutical composition is in the form of a spray.

In one embodiment, the acneiform lesions are induced by an EGFR inhibitor, RAS inhibitor, ERK inhibitor, SHP inhibitor, MEK inhibitor, and/or HER inhibitor. In one embodiment, the subject in need thereof is a subject has a cancer treated with EGFR inhibitor, RAS inhibitor, ERK inhibitor, SHP inhibitor, PI3K inhibitor, MEK inhibitor and/or HER dimerization inhibitor.

In one embodiment, the subject in need thereof has grade 1 acneiform lesions. In one embodiment, the subject in need thereof has grade 2 acneiform lesions. In one embodiment, the subject in need thereof has grade 3 acneiform lesions. In one embodiment, the subject in need thereof has grade 4 acneiform lesions.

In one embodiment, the method reduces the severity or prevents escalation of the acneiform lesions. In one embodiment, the method reduces the severity of the acneiform lesions from grade 4 to grade 3, 2, 1, or 0. In one embodiment, the method reduces the severity of the acneiform lesions from grade 3 to grade 2, 1, or 0. In one embodiment, the method reduces the severity of the acneiform lesions from grade 2 to grade 1, or 0. In one embodiment, the method reduces the severity of the acneiform lesions from grade 1 to grade 0.

In one embodiment, the acneiform lesions are induced by treatment with gefitinib, erlotinib, lapatinib, cetuximab, panitumumab, vandetanib, necitumumab, osimertinib, afatinib, neratinib, pertuzumab, amivantamab, lazertinib or combinations thereof.

In one embodiment, the acneiform lesions are induced by treatment with Sotorasib, Adagrasib, RMC-6291, JDQ443 or combinations thereof.

In one embodiment, the acneiform lesions are induced by treatment with SHP141, SHP099, RMC-4630, ERAS-601 or combinations thereof.

In one embodiment, the acneiform lesions are induced by treatment with Cobimetinib, binimetinib, Selumetinib, trametinib or combinations thereof.

In one embodiment, the acneiform lesions are induced by treatment with: Temuterkib, Ulixertinib, ERAS-007 or combinations thereof.

In one embodiment, compositions for use in the treatment and/or prevention of EGFR inhibitors, PI3K inhibitors, MEK inhibitors, RAS inhibitors, SHP inhibitors, ERK inhibitors and/or HER dimerization inhibitors-induced cutaneous lesions are provided, wherein the composition is topically administered to the skin of a subject in need thereof on a daily basis for 4 to 12 weeks, and the composition comprises from 0.01% to 1% w/w, from 0.01% to 0.1% w/w, from 0.01% to 0.3% w/w, from 0.03% to 1% w/w, from 0.03% to 0.3% w/w, from 0.03% to 0, 1% w/w of the compound of Formula I. In one embodiment the cutaneous lesions are acneiform lesions.

In one embodiment, compositions for use in the treatment and/or prevention of EGFR inhibitors, PI3K inhibitors, MEK inhibitors, RAS inhibitors, SHP inhibitors, ERK inhibitors and/or HER dimerization inhibitors induced cutaneous lesions are provided, wherein the composition is topically administered to the skin of a subject in need thereof twice daily for 4 to 12 weeks, and the composition comprises from 0.01% to 1% w/w, from 0.01% to 0.1% w/w, from 0.01% to 0.3% w/w, from 0.03% to 1% w/w, from 0.03% to 0.3% w/w, from 0.03% to 0.25% w/w, from 0.03% to 0.1% w/w of the compound of Formula I. In one embodiment the cutaneous lesions are acneiform lesions. In one embodiment, the acneiform lesions are induced by an EGFR inhibitor. In one embodiment, the acneiform lesions are induced by treatment with gefitinib, erlotinib, lapatinib, cetuximab, panitumumab, vandetanib, necitumumab, osimertinib, afatinib, neratinib, pertuzumab or combinations thereof.

In one embodiment, the subject in need thereof is a subject has a cancer treated with RAS inhibitor, SHP inhibitors, ERK inhibitors, PI3K inhibitor, MEK inhibitor and/or HER dimerization inhibitor.

In one embodiment, the subject in need thereof has grade 1 acneiform lesions. In one embodiment, the subject in need thereof has grade 2 acneiform lesions. In one embodiment, the subject in need thereof has grade 3 acneiform lesions. In one embodiment, the subject in need thereof has grade 4 acneiform lesions.

In one embodiment, the composition reduces the severity or prevents escalation of the acneiform lesions. In one embodiment, the composition reduces the severity of the acneiform lesions from grade 4 to grade 3, 2, 1, or 0. In one embodiment, the method reduces the severity of the acneiform lesions from grade 3 to grade 2, 1, or 0. In one embodiment, the composition reduces the severity of the acneiform lesions from grade 2 to grade 1, or 0. In one embodiment, the composition reduces the severity of the acneiform lesions from grade 1 to grade 0.

### EXAMPLES

The following examples illustrate certain embodiments of the invention but are not meant to limit the scope of the claims in any way. The following examples are put forth so as to provide those of ordinary skill in the art with a complete disclosure and description of how to make and use the described invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Use of BRAF Inhibitors to Treat a RAS Inhibitor-Induced Cutaneous Adverse Reaction in a Subject in Need Thereof

Treat cancer subjects diagnosed with RAS inhibitor-associated cutaneous adverse reactions with a therapeutically effective amount of a composition comprising at least one BRAF inhibitor of Formula I-IV so as to determine their in vivo efficacy. A placebo treated group (the vehicle) is included as a negative control.

The treatment protocol includes the following steps:

Subjects are patients that have been treated with a RAS inhibitor and have been diagnosed with cutaneous adverse reactions (such as acneiform rash). The subjects are treated for 4-20 weeks with said BRAF inhibitor composition in a topical gel dosage form, on the affected skin parts like face, neck and chest. Different concentrations of the BRAF inhibitor will be tested and compared. Control subjects are treated with a suitable placebo composition, which does not include a BRAF inhibitor. When applied topically, the placebo treatment is performed on the same body areas of control patients. During the treatment period, various parameters are tested, including, the rate of the cutaneous condition eruptions, the severity of the eruptions, the body location of the eruptions and other related cutaneous events. The body locations tested will include the face, neck and chest.

At the end of the trial treatment period, the tested parameters are determined by comparing the treated body regions with those of the placebo-treated patients.

It is anticipated that the patients treated with the BRAF inhibitor will show amelioration in the rate and/or severity of eruptions and/or other cutaneous events compared to that observed with the placebo-treated patients. Accordingly, the topical BRAF inhibitor compositions described herein are useful for treating or preventing RAS inhibitor-associated cutaneous conditions in a subject in need thereof.

### Prophetical

### Example 2: Topical Treatment, Prevention of Escalation and/or Reduction of the Severity of a Cutaneous Adverse Reaction in a Subject by Concomitant Administration of a First Composition Comprising a Therapeutically Effective Dose of a Compound Comprising Formula I, Formula II, Formula III, or Formula IV Disclosed Herein

Subjects having an adverse cutaneous reaction are treated with a topical composition comprising from 0.01% weight/weight to 0.3% weight/weight of a compound comprising Formula I, Formula II, Formula III, or Formula IV as disclosed herein. As a control, a placebo treatment (vehicle) is provided to control treated subjects.

The subjects are treated with the topical composition at least once daily for four to six weeks to the adverse cutaneous reaction (e.g., skin lesions) of the subjects. Control subjects are treated with a corresponding suitable placebo composition, which does not include a compound comprising Formula I, Formula II, Formula III, or Formula IV as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 3: Topical Treatment, Prevention of Escalation and/or Reduction of the Severity of an Adverse Cutaneous Reaction in a Subject by Concomitant Administration of a First Composition Comprising a Therapeutically Effective Dose of Compound Comprising Formula I

Subjects having an adverse cutaneous reaction are treated with a topical composition comprising from 0.01% weight/weight to 0.3% weight/weight of a compound comprising Formula I. As a control, a placebo treatment (vehicle) is provided to control treated subjects.

The subjects are treated with the topical composition at least once daily for four to six weeks to the adverse cutaneous reaction (e.g., skin lesions) of the subjects. Control subjects are treated with a corresponding suitable placebo composition, which does not include Formula I as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 4: Topical Treatment, Prevention of Escalation and/or Reduction of the Severity of an Adverse Cutaneous Reaction in a Subject by Concomitant Administration of a First Composition Comprising a Therapeutically Effective Dose of a Compound Comprising Formula II

Subjects having an adverse cutaneous reaction (e.g., cutaneous lesions) are treated with a topical composition comprising from 0.01% weight/weight to 0.3% weight/weight of a compound comprising Formula II as disclosed herein. As a control, a placebo treatment (vehicle) is provided to control treated subjects.

The subjects are treated with the topical composition at least once daily for four to six weeks to the adverse cutaneous reaction (e.g., skin lesions) of the subjects. Control subjects are treated with a corresponding suitable placebo composition, which does not include Formula II as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 5: Topical Treatment, Prevention of Escalation and/or Reduction of the Severity of an Adverse Cutaneous Reaction in a Subject by Concomitant Administration of a First Composition Comprising a Therapeutically Effective Dose of a Compound Comprising Formula III

Subjects having an adverse cutaneous reaction are treated with a topical composition comprising from 0.01% weight/weight to 0.3% weight/weight of a compound comprising Formula III as disclosed herein. As a control, a placebo treatment (vehicle) is provided to control treated subjects.

The subjects are treated with the topical composition at least once daily for four to six weeks to the adverse cutaneous reaction (e.g., skin lesions) of the subjects. Control subjects are treated with a corresponding suitable placebo composition, which does not include Formula III as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 6: Topical Treatment, Prevention of Escalation and/or Reduction of the Severity of an Adverse Cutaneous Reaction in a Subject by Concomitant Administration of a First Composition Comprising a Therapeutically Effective Dose of Compound Comprising Formula IV

Subjects having an adverse cutaneous reaction are treated with a topical composition comprising from 0.01% weight/weight to 0.3% weight/weight of a compound comprising Formula IV. As a control, a placebo treatment (vehicle) is provided to control treated subjects.

The subjects are treated with the topical composition at least once daily for four to six weeks to the adverse cutaneous reaction (e.g., skin lesions) of the subjects. Control subjects are treated with a corresponding suitable placebo composition, which does not include Formula IV as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 7: Topical Treatment, Prevention of Escalation and/or Reduction of the Severity of an Adverse Cutaneous Reaction in a Subject by Concomitant Administration of a First Composition Comprising a Therapeutically Effective Dose of a Compound Comprising Formula I and Formula II

Subjects having an adverse cutaneous reaction are treated with a topical composition comprising from 0.01% weight/weight to 0.3% weight/weight of a compound comprising Formula I and Formula II as disclosed herein. As a control, a placebo treatment (vehicle) is provided to control treated subjects.

The subjects are treated with the topical composition at least once daily for four to six weeks to the adverse cutaneous reaction of the subjects. Control subjects are treated with a corresponding suitable placebo composition, which does not include Formula I or Formula II as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 8: Topical Treatment, Prevention of Escalation and/or Reduction of the Severity of an Adverse Cutaneous Reaction in a Subject by Concomitant Administration of a First Composition Comprising a Therapeutically Effective Dose of a Compound Comprising Formula I and Formula III

Subjects having an adverse cutaneous reaction are treated with a topical composition comprising from 0.01% weight/weight to 0.3% weight/weight of a compound comprising Formula I and Formula III. As a control, a placebo treatment (vehicle) is provided to control treated subjects.

The subjects are treated with the topical composition at least once daily for four to six weeks to the adverse cutaneous reaction of the subjects. Control subjects are treated with a corresponding suitable placebo composition, which does not include Formula I or Formula III as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 9: Topical Treatment, Prevention of Escalation and/or Reduction of the Severity of an Adverse Cutaneous Reaction in a Subject by Concomitant Administration of a First Composition Comprising a Therapeutically Effective Dose of a Compound Comprising Formula II and Formula III

Subjects having an adverse cutaneous reaction are treated with a topical composition comprising from 0.01% weight/weight to 0.3% weight/weight of a compound comprising Formula II and Formula III. As a control, a placebo treatment (vehicle) is provided to control treated subjects.

The subjects are treated with the topical composition at least once daily for four to six weeks to the adverse cutaneous reaction of the subjects. Control subjects are treated with a corresponding suitable placebo composition, which does not include Formula II or Formula III as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 10: Treating Cutaneous Lesions in a Subject Having an Adverse Cutaneous Reaction with a Systemically Administered Composition Comprising a Therapeutically Effective Dose of a Compound Comprising Formula I, Formula II, Formula III, or Formula IV as Disclosed Herein

To treat cutaneous conditions induced or caused by an adverse cutaneous reaction, subjects that have been diagnosed with such cutaneous conditions (i.e., subjects that have an adverse cutaneous reaction) are systemically administered with a composition comprising Formula I, Formula II, Formula III, or Formula IV to treat said condition. As a control, a placebo treatment (vehicle) is provided to control treated subjects.

Briefly, the treatment protocol includes the following steps:
Subjects—Patients having cutaneous lesions caused by an adverse cutaneous reaction.
Treatment—the subjects are treated for 6-20 weeks with a systemic composition comprising a compound comprising Formula I, Formula II, Formula III, or Formula IV in a suitable dosage form. The dosage form is an oral dosage form comprising 200-1000 micrograms of the compound.
Control—subjects are treated with a corresponding suitable placebo composition, which does not include Formula I, Formula II, Formula III, or Formula IV as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 11: Treating Cutaneous Lesions in a Subject Having an Adverse Cutaneous Reaction with a Systemically Administered Composition Comprising a Therapeutically Effective Dose of a Compound Comprising Formula I as Disclosed Herein

To treat cutaneous conditions induced or caused by an adverse cutaneous reaction, subjects that have been diagnosed with such cutaneous conditions (i.e., subjects that have an adverse cutaneous reaction) are systemically administered with a composition comprising Formula I to treat said condition. As a control, a placebo treatment (vehicle) is provided to control treated subjects.

Briefly, the treatment protocol includes the following steps:
Subjects—Patients having cutaneous lesions caused by an adverse cutaneous reaction.
Treatment—the subjects are treated for 6-20 weeks with a systemic composition comprising a compound comprising Formula I in a suitable dosage form. The dosage form is an oral dosage form comprising 200-1000 micrograms of the compound.
Control—subjects are treated with a corresponding suitable placebo composition, which does not include Formula I as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 12: Treating Cutaneous Lesions in a Subject Having an Adverse Cutaneous Reaction with a Systemically Administered Composition Comprising a Therapeutically Effective Dose of a Compound Comprising Formula II as Disclosed Herein

To treat cutaneous conditions induced or caused by an adverse cutaneous reaction, subjects that have been diagnosed with such cutaneous conditions (i.e., subjects that have an adverse cutaneous reaction) are systemically administered with a composition comprising Formula II to treat said condition. As a control, a placebo treatment (vehicle) is provided to control treated subjects.

Briefly, the treatment protocol includes the following steps:
Subjects—Patients having cutaneous lesions caused by an adverse cutaneous reaction.
Treatment—the subjects are treated for 6-20 weeks with a systemic composition comprising a compound comprising Formula II in a suitable dosage form. The dosage form is an oral dosage form comprising 200-1000 micrograms of the compound.
Control—subjects are treated with a corresponding suitable placebo composition, which does not include Formula II as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 13: Treating Cutaneous Lesions in a Subject Having an Adverse Cutaneous Reaction with a Systemically Administered Composition Comprising a Therapeutically Effective Dose of a Compound Comprising Formula III as Disclosed Herein

To treat cutaneous conditions induced or caused by an adverse cutaneous reaction, subjects that have been diagnosed with such cutaneous conditions (i.e., subjects that have an adverse cutaneous reaction) are systemically administered with a composition comprising Formula III to treat said condition. As a control, a placebo treatment (vehicle) is provided to control treated subjects.

Briefly, the treatment protocol includes the following steps:
Subjects—Patients having cutaneous lesions caused by an adverse cutaneous reaction.
Treatment—the subjects are treated for 6-20 weeks with a systemic composition comprising a compound comprising Formula III in a suitable dosage form. The dosage form is an oral dosage form comprising 200-1000 micrograms of the compound.
Control—subjects are treated with a corresponding suitable placebo composition, which does not include Formula III as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 14: Treating Cutaneous Lesions in a Subject Having an Adverse Cutaneous Reaction with a Systemically Administered Composition Comprising a Therapeutically Effective Dose of a Compound Comprising Formula IV as Disclosed Herein

To treat cutaneous conditions induced or caused by an adverse cutaneous reaction, subjects that have been diagnosed with such cutaneous conditions (i.e., subjects that have an adverse cutaneous reaction) are systemically administered with a composition comprising Formula IV to treat said condition. As a control, a placebo treatment (vehicle) is provided to control treated subjects.

Briefly, the treatment protocol includes the following steps:
Subjects—Patients having cutaneous lesions caused by an adverse cutaneous reaction.
Treatment—the subjects are treated for 6-20 weeks with a systemic composition comprising a compound comprising Formula IV in a suitable dosage form. The dosage form is an oral dosage form comprising 200-1000 micrograms of the compound.
Control—subjects are treated with a corresponding suitable placebo composition, which does not include Formula IV as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 15: Treating Cutaneous Lesions in a Subject Having an Adverse Cutaneous Reaction with a Systemically Administered Composition Comprising a Therapeutically Effective Dose of a Compound Comprising Formula I and II as Disclosed Herein

To treat cutaneous conditions induced or caused by an adverse cutaneous reaction, subjects that have been diagnosed with such cutaneous conditions (i.e., subjects that have an adverse cutaneous reaction) are systemically administered with a composition comprising Formula I and Formula II to treat said condition. As a control, a placebo treatment (vehicle) is provided to control treated subjects.

Briefly, the treatment protocol includes the following steps:
Subjects—Patients having cutaneous lesions caused by an adverse cutaneous reaction.
Treatment—the subjects are treated for 6-20 weeks with a systemic composition comprising a compound comprising Formula I and Formula II in a suitable dosage form. The dosage form is an oral dosage form comprising 200-1000 micrograms of the compound.
Control—subjects are treated with a corresponding suitable placebo composition, which does not include Formula I or Formula II as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 16: Treating Cutaneous Lesions in a Subject Having an Adverse Cutaneous Reaction with a Systemically Administered Composition Comprising a Therapeutically Effective Dose of a Compound Comprising Formula I and III as Disclosed Herein

To treat cutaneous conditions induced or caused by an adverse cutaneous reaction, subjects that have been diagnosed with such cutaneous conditions (i.e., subjects that have an adverse cutaneous reaction) are systemically administered with a composition comprising Formula I and Formula III to treat said condition. As a control, a placebo treatment (vehicle) is provided to control treated subjects.

Briefly, the treatment protocol includes the following steps:
Subjects—Patients having cutaneous lesions caused by an adverse cutaneous reaction.
Treatment—the subjects are treated for 6-20 weeks with a systemic composition comprising a compound comprising Formula I and Formula III in a suitable dosage form. The dosage form is an oral dosage form comprising 200-1000 micrograms of the compound.
Control—subjects are treated with a corresponding suitable placebo composition, which does not include Formula I or Formula III as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical

### Example 17: Treating Cutaneous Lesions in a Subject Having an Adverse Cutaneous Reaction with a Systemically Administered Composition Comprising a Therapeutically Effective Dose of a Compound Comprising Formula II and III as Disclosed Herein

To treat cutaneous conditions induced or caused by an adverse cutaneous reaction, subjects that have been diagnosed with such cutaneous conditions (i.e., subjects that have an adverse cutaneous reaction) are systemically administered with a composition comprising Formula II and Formula III to treat said condition. As a control, a placebo treatment (vehicle) is provided to control treated subjects.

Briefly, the treatment protocol includes the following steps:
Subjects—Patients having cutaneous lesions caused by an adverse cutaneous reaction.
Treatment—the subjects are treated for 6-20 weeks with a systemic composition comprising a compound comprising Formula II and Formula III in a suitable dosage form. The dosage form is an oral dosage form comprising 200-1000 micrograms of the compound.
Control—subjects are treated with a corresponding suitable placebo composition, which does not include Formula II or Formula III as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### EMBODIMENTS

In one embodiment, there is provided a method of treatment, prevention of escalation and/or reduction of the severity of adverse cutaneous reaction in a subject in need thereof, comprising administration to the skin of a subject in need thereof a therapeutically effective amount of a topical pharmaceutical composition comprising a compound of Formula I,

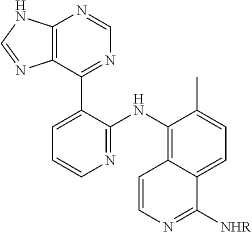

wherein R is selected from the group consisting of 3-ethynylphenyl, 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, 3,5-dihydroxyphenyl or phenyl-3-sulfonamide, or a pharmaceutically acceptable salt or a solvate thereof, or a compound of Formula II

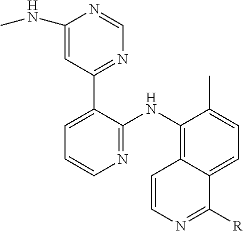

wherein R is NHR¹, wherein R¹ is 2-fluoro-4-iodophenyl, or a pharmaceutically acceptable salt or a solvate thereof, or a compound of Formula III

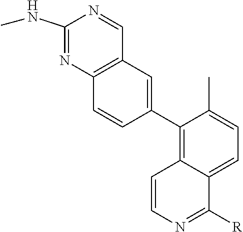

wherein R is NHR¹, wherein R¹ is 3-ethynylphenyl, 3-chloro-4-fluorophenyl, 2-fluoro-4-iodophenyl, or 4-chloro-3-(trifluoromethyl)phenyl, or a pharmaceutically acceptable salt or a solvate thereof, or a compound of Formula IV

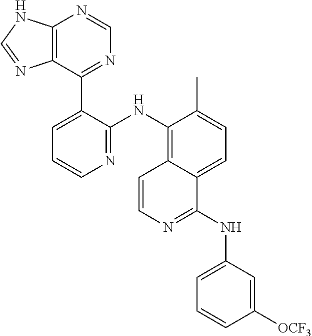 [SMILES: Cc1ccc2c(Nc3cccc(OC(F)(F)F)c3)nccc2c1Nc1ncccc1-c1ncnc2[nH]cnc12]

and a pharmaceutically acceptable carrier or excipient.

In one embodiment, there is provided a method of treatment, prevention of escalation and/or reduction of the severity of an adverse cutaneous reaction in a subject in need thereof, wherein the treatment comprises administration to the skin of the subject a topical composition comprising a therapeutically effective dose of at least one compound of Formula I-IV.

In one embodiment, there is provided a method of treatment, prevention of escalation and/or reduction of the severity of an adverse cutaneous reaction in a subject in need thereof, wherein the adverse cutaneous reaction subject has an adverse cutaneous reaction accompanied by cutaneous lesions, wherein the method comprises concomitant administration of a topical composition comprising a therapeutically effective dose of at least one compound of Formula I-IV and a therapeutically effective dose of a drug or treatment selected from: a. oral vemurafenib b. any other regulatory approvable BRaf inhibitor effective for systemic adverse cutaneous reaction treatment or c. any other regulatory approvable treatment effective for systemic adverse cutaneous reaction treatment.

In one embodiment, there is provided a method of treatment, prevention of escalation and/or reduction of the severity of an adverse cutaneous reaction in a subject in need thereof, wherein the method comprises concomitant administration of a topical composition comprising a therapeutically effective dose of at least one compound of Formula I-IV and a therapeutically effective dose of an oral second treatment.

In one embodiment, there is provided a method of treatment, prevention of escalation or reduction of the severity of cutaneous lesions in a subject, comprising administration to the skin of the subject a therapeutically effective amount of a topical pharmaceutical composition comprising a compound of Formula I-IV, wherein the topical pharmaceutical composition is applied for four to six weeks to the adverse cutaneous reaction skin lesions of the subject in need thereof.

In one embodiment, there is provided a method of treatment, prevention of escalation or reduction of the severity of cutaneous lesions in a subject, comprising administration to the skin of the subject a therapeutically effective amount of a topical pharmaceutical composition comprising a compound of Formula I-IV, wherein the topical pharmaceutical composition is in the form of a gel, a hydrogel, an ointment, a cream, a spray, a dermal patch, a foam, a lotion or a liquid.

In one embodiment, there is provided a method of treatment, prevention of escalation or reduction of the severity of cutaneous lesions in a subject in need thereof, comprising administration to the skin of the subject a therapeutically effective amount of a topical pharmaceutical composition comprising a therapeutically-effective amount of a compound of Formula I-IV, wherein the topical pharmaceutical composition is in the form of a gel.

In one embodiment, there is provided a method of treatment, prevention of escalation or reduction of the severity of cutaneous lesions in an adverse cutaneous reaction subject in need thereof, comprising administration to the skin of the subject a therapeutically effective amount of a topical pharmaceutical composition comprising a compound of Formula I, II, III or IV, wherein the topical pharmaceutical composition is in the form of a spray.

In one embodiment, there is provided a method of treatment, prevention of escalation or reduction of the severity of cutaneous lesions in a subject in need thereof, comprising administration to the skin of the subject a therapeutically effective amount of a topical pharmaceutical composition comprising a compound of Formula I-IV, wherein the subject in need thereof has an adverse cutaneous reaction disease.

In one embodiment, there is provided a method of treatment, prevention of escalation or reduction of the severity of cutaneous lesions in a subject in need thereof, comprising administration to the skin of the subject a therapeutically effective amount of a topical pharmaceutical composition comprising a compound of Formula I, II, III or IV, wherein the method reduces the severity or prevents escalation of the adverse cutaneous reaction.

In one embodiment, there is provided a topical pharmaceutical composition comprising from 0.01% to 1.00% weight/weight of the compound of Formula IV.

In one embodiment, there is provided a topical pharmaceutical composition comprising from 0.01% weight/weight to 0.3% weight/weight of a compound of Formula IV:

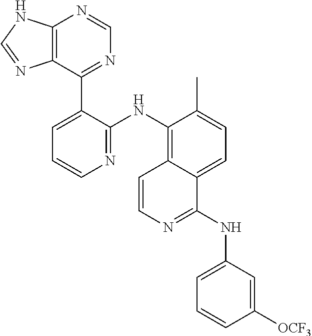 [SMILES: Cc1ccc2c(Nc3cccc(OC(F)(F)F)c3)nccc2c1Nc1ncccc1-c1ncnc2[nH]cnc12]

and a pharmaceutically acceptable carrier or excipient.

In one embodiment, there is provided a topical composition, comprising a therapeutically-effective amount of a compound of Formula I-IV, wherein the composition is in the form of a gel, a hydrogel, an ointment, a cream, a foam, a spray, a lotion, a liquid or a dermal patch.

In one embodiment, there is provided a topical composition, comprising a therapeutically-effective amount of a compound of Formula I-IV, wherein the composition comprising from 0.01 to 0.3% weight/weight of the compound of Formula IV.

In one embodiment, there is provided a topical composition, comprising a compound of Formula I-IV, wherein the composition comprising about 0.03% weight/weight of the compound of Formula IV.

In one embodiment, there are provided pharmaceutical topical compositions, comprising a therapeutically-effective amount of at least one compound of Formula I-IV and methods of using pharmaceutical topical compositions comprising a compound of Formula I-IV for the treatment, prevention of adverse cutaneous reactions including escalation and reduction of the severity of adverse cutaneous reactions.

What is claimed is:

1. A method of treating, ameliorating and/or preventing an EGFR, RAS, RAF, ERK, PI3K, MEK, SHP or HER inhibitor-induced cutaneous adverse reaction (each alone or in combination with another inhibitor) in a subject in need thereof, comprising:
administering to the subject a topical pharmaceutical composition comprising a therapeutically effect amount of a compound of Formula I-IV and a pharmaceutically acceptable carrier or excipient to the subject for treating said inhibitor-induced cutaneous adverse reactions, wherein one symptom of the inhibitor-induced cutaneous adverse reaction is one or more acneiform lesions,
wherein said treatment improves adherence of the subject to the treatment and prevents delays, discontinuation or interruption of the treatment or undesirable reduction of the label recommended inhibitor dosage,
wherein the compounds of Formula I-IV are effective for prevention,
amelioration or treatment of cutaneous adverse effects at all MAPK pathway levels,
wherein the treatment is successful in ameliorating the severity of the acneiform lesion by at least by one unit, and
wherein said treatment is successful in preventing escalation of the cutaneous adverse effects.

2. The method of claim 1, wherein the acneiform lesions are EGFR, RAS, ERK, PI3K, MEK, SHP or HER inhibitor (each alone or in combination with another inhibitor), -induced acneiform lesions.

3. The method of claim 1, wherein the step of administration comprises applying the topical pharmaceutical composition for four to twelve weeks to the skin of the subject in need thereof.

4. The method of claim 1, wherein the topical pharmaceutical composition is in the form of a gel, a hydrogel, an ointment, a cream, a spray, a dermal patch, a foam, a lotion or a liquid.

5. The method of claim 1, wherein the topical pharmaceutical composition is in the form of a gel.

6. The method of claim 1, wherein the topical pharmaceutical composition is in the form of a spray.

7. The method of claim 1, wherein the subject in need thereof has a cancer treated with an RAS or ERK or SHP inhibitor.

8. The method of claim 1, wherein the subject in need thereof has grade 1 acneiform lesions.

9. The method of claim 1, wherein the subject in need thereof has grade 2 acneiform lesions.

10. The method of claim 1, wherein the subject in need thereof has grade 3 acneiform lesions.

11. The method of claim 1, wherein the subject in need thereof has grade 4 acneiform lesions.

12. The method of claim 1, wherein the method reduces the severity or prevents escalation of the acneiform lesions.

13. The method of claim 1, wherein the acneiform lesions are induced by treatment with gefitinib, erlotinib, lapatinib, cetuximab, panitumumab, vandetanib, necitumumab, osimertinib, afatinib, neratinib, pertuzumab or combinations thereof.

14. The method of claim 1, wherein the topical pharmaceutical composition comprises from about 0.03 to about 0.15% weight/weight of the compound of Formula I.

15. The method of claim 1, wherein the topical pharmaceutical composition comprises from about 0.01% weight/weight to about 0.2% w/w of the compound of Formula I.

16. A topical pharmaceutical composition comprising from about 0.03% weight/weight to about 0.1% weight/weight of a compound of Formula I and a pharmaceutically acceptable carrier or excipient.

17. The topical pharmaceutical composition of claim 16, wherein the composition is in the form of a gel, a hydrogel, an ointment, a cream, a foam, a spray, a lotion, a liquid or a dermal patch.

18. The topical pharmaceutical composition of claim 16, for use in a method of treating and/or preventing RAS inhibitor-induced acneiform lesions in a subject in need thereof.

19. The topical pharmaceutical composition of claim 16 for use in a method of treating and/or preventing an EGFR, RAS, ERK, PI3K, MEK, SHP or HER inhibitor (each alone or in combination with another inhibitor)-induced acneiform lesions in a subject in need thereof, wherein the topical pharmaceutical composition is administered topically for four to twelve weeks to the skin of the subject in need thereof.

20. The method of claim 1, wherein the compound comprises Formula IV.
